(19) United States
(12) Patent Application Publication
(10) Pub. No.: US 2025/0182277 A1
(43) Pub. Date: Jun. 5, 2025

(54) METHOD FOR PROCESSING THREE-DIMENSIONAL MEDICAL INPUT IMAGE DATA AND FOR PARAMETERIZING AN ESTABLISHING ALGORITHM, DATA PROCESSING FACILITY AND COMPUTER PROGRAM

(71) Applicant: Siemens Healthineers AG, Forchheim (DE)

(72) Inventor: Matthias FENCHEL, Bubenreuth (DE)

(73) Assignee: Siemens Healthineers AG, Forchheim (DE)

(21) Appl. No.: 18/962,077

(22) Filed: Nov. 27, 2024

(30) Foreign Application Priority Data
Nov. 30, 2023 (DE) 10 2023 211 990.0

(51) Int. Cl. G06T 7/00; G06T 3/40; G06T 15/00

(52) U.S. Cl. CPC G06T 7/0012; G06T 3/40; G06T 15/00; G06T 2207/10088; G06T 2207/30104

(57) ABSTRACT

A computer-implemented method comprises: obtaining input image data that defines image values for a first number of input image voxels; establishing a respective vesselness measure for at least parts of the input image voxels, wherein the respective vesselness measure defines an established degree of similarity of a structure mapped in a respective input image voxel to a mapping of a vessel; and applying a filter algorithm to the input image data for providing filtered image data, or scaling the image values of the input image voxels for providing scaled image data. A parameter value of at least one filter parameter of the filter algorithm is dependent upon at least one vesselness measure, or a respective scaling factor used for scaling a respective image value is dependent upon at least one vesselness measure.

### CROSS-REFERENCE TO RELATED APPLICATION(S)

The present application claims priority under 35 U.S.C. § 119 to German Patent Application No. 10 2023 211 990.0, filed Nov. 30, 2023, the entire contents of which is incorporated herein by reference.

### FIELD

One or more embodiments of the present invention relate to a computer-implemented method for processing three-dimensional medical input image data. In addition, one or more embodiments of the present invention relate to a method for parameterizing an establishing algorithm, a data processing device (or facility) and a non-transitory computer-readable storage medium storing a computer program.

### BACKGROUND

In the context of medical imaging, an image quality that is achievable can be limited by properties of the imaging apparatus being used. In order to reduce a burden on a patient, it can also be desirable, for example, to use very low X-ray doses and/or as far as possible, to dispense with contrast media during imaging. Although in some imaging methods, for example, in many magnetic resonance protocols, a contrast improvement and/or a noise reduction can be achieved with longer scan times, long scan times reduce the tolerability of an investigation, increase its costs and can also be disadvantageous for the achievable image quality, due to movement artifacts. In many application cases, it is therefore essential to enhance the image quality sufficiently with a suitable post-processing and/or filtration of the image data, in order to enable a robust evaluation.

Such a post-processing is particularly relevant for low-field magnetic resonance scanning, in particular, when scans are to be carried out with a magnetic field of less than one tesla. Although low-field scans are possible with significantly more economical scanning facilities than other magnetic resonance scans and thus, in particular, can significantly improve the local availability of magnetic resonance scans, it is inherent therein that lower signal-to-noise ratios are achieved. Thus, for example, a robust magnetic resonance angiography is typically only possible in the low-field domain by using contrast media.

Since, however, a magnetic resonance angiography with a low field strength would, for example, be particularly advantageous, in order to carry out particular preventative check-ups in rural regions, it would be advantageous also in the low-field domain to be able to use contrast medium-free scanning methods for angiography, for example, ASL (arterial spin labeling) or time-of-flight methods.

Since, however, conventional methods for contrast improvement and noise suppression and/or background suppression in the aforementioned field of use have their limits, the development of further approaches for improving the image quality is suitable.

A possible approach for this is the use of algorithms trained by machine learning, in particular deep learning, for noise suppression. Since, however, the provision of such an algorithm can be complex, in particular since, for a use in the context of the medical imaging, the risk of a corruption of the image by such an algorithm must be minimized, a development of other approaches for post-processing of medical image data and/or a further optimization of approaches based upon machine learning is however suitable.

### SUMMARY

Independent of the grammatical term usage, individuals with male, female or other gender identities are included within the term.

It is an object of one or more embodiments of the present invention to provide an improved approach to the quality improvement of medical image data, in particular, image data from a magnetic resonance angiography, specifically at low field strengths, and/or for background suppression, in particular, for an angiography.

At least this object is achieved by way of a computer-implemented method for processing three-dimensional medical input image data, which comprises the following steps:
obtaining the input image data which defines image values for a large number of input image voxels,
establishing a respective vesselness measure for at least parts of the input image voxels, wherein the vesselness measure defines a degree of the similarity of a structure mapped in the respective input image voxel to a vessel, and
firstly, applying a filter algorithm to the input image data for providing filtered image data, wherein the parameter value of at least one filter parameter of the filter algorithm is specified dependent upon respectively at least one of the vesselness measures, or
secondly, scaling the image values of the input image voxels for providing scaled image data, wherein a respective scaling factor used for scaling the respective input image voxel is specified dependent, in each case, upon at least one of the vesselness measures.

The relevant structures mapped in the context of particular medical imaging processes, in particular, in the context of an angiography are at least approximately vessel-like and are thus formed, in particular, tubular and/or cylindrical and are thus mapped and/or localized by way of the vesselness measures used according to an embodiment of the present invention, which are able, in particular, to form a three-dimensional vesselness map.

By way of a mutually different scaling of the image values of different input image voxels dependent upon the respective local vesselness measure, for example, input image voxels with a value of the vesselness measure above a particular limit value can be scaled with a higher scaling factor, than input image voxels, the vesselness measure of which falls below the limit value. Alternatively, for example, for the respective input image voxel, a scaling of its image value that is proportional to the value of the vesselness measure established for this input image voxel can take place. By way of the scaling dependent upon the local vesselness measure, for example, a background can thus be suppressed and thus a mapped vessel tree can be emphasized.

By way of the parameterization of the filter algorithm dependent upon the vesselness measure, for example, in the vessel region and outside the vessel region, that is for example, for input image voxels with a value of the vesselness measure above or below a limit value, different-strength filtrations can be carried out. The parameter value of the filter parameter or at least one of the filter parameters can thus specify the strength of the filtration.

For example, in the vessel region a narrower filter kernel, for example, a Gaussian curve with a lower variance can be used than outside the vessel region. The filter parameter or a respective parameter of the filter parameters can thus specify a width of the filter kernel, for example, the variance of a Gaussian kernel, and/or a form of the filter kernel. A stronger filtration and/or blurring of the background region as compared with a vessel tree facilitates the analysis of the vessel tree since disturbances in the background are suppressed without blurring the regions of the vessel tree itself too strongly. Apart from a quality improvement, by way of strong filtration and/or a suitable selection of the filter parameters in the background region in addition, or alternatively, to the aforementioned scaling, a background suppression can be achieved wherein background structures, specifically relatively large background structures with which an observer can orientate himself, can further remain visible.

For example, an anisotropic diffusion filter or suchlike can be used and, dependent upon the vesselness measure, can be parameterized differently locally in order to achieve the effects described. In particular, if the filter algorithm is an anisotropic filter algorithm, the filter parameters or at least one of the filter parameters, as described in more detail below, can depend also upon the direction in which the respective recognized vessel-like structure extends. By this mechanism and/or means, for example, in the longitudinal direction of the vessel-like structure, a stronger filtration can take place than perpendicularly thereto.

Additionally or alternatively, for scaling the image values of the input image voxels of the input image data dependent upon the vesselness measure, a corresponding scaling can also take place for the filtered image data or the scaled image data can be filtered, as described in greater detail below.

For the establishment of suitable vesselness measures, for example, the algorithm identified as a “line filter” in the publication by Sato, Y et al. (1997), 3D multi-scale line filter for segmentation and visualization of curvilinear structures in medical images, in Troccaz, J., Grimson, E., Mösges, R. (eds) CVRMed-MRCAS' 97. CVRMed MRCAS 1997 1997, Lecture Notes in Computer Science, vol 1205, Springer, Berlin, Heidelberg. https://doi.org/10.1007/BFb0029240, can be applied to the input image data or to an intermediate result determined by interpolation of the input image data, for instance.

In order to calculate the vesselness measure, initially a value matrix associated with the respective input image voxel can be established. This value matrix can be established, in particular, by way of a convolution of the Hessian matrix of a Gaussian kernel G or another filter kernel with the input image data. The entries to the value matrix can be given, in particular, as

$${I}_{ab} ( \vec{x} , σ ) = \{ \frac{δ}{δ a} \frac{δ}{δ b} G ( \vec{x} , σ ) \} * I ( \vec{x} )$$

wherein I specifies the image value of the input image voxel at the position specified by the position vector \vec{x}. Iab is the respective matrix entry for this input image voxel, wherein a, b ϵ {x, y, z} defines the derivative into the respective spatial direction and the different matrix entries Iab are associated with different combinations and/or sequences of the derivations.

The variance σ of the Gaussian distribution G selects the diameter of the tubular or cylindrical structures for which the establishment of the vesselness is to be selective. In this way, σ can be selected by the user, for example in the context of the image optimization, dynamically by way of a controller or suchlike, or automatically, for example by way of the multiscale approach mentioned below.

From the value matrix, three eigenvalues λ1, λ2, λ3 can then be calculated for the respective input image voxel. For linear structures, the eigenvalue for the eigenvector in the longitudinal direction of the vessel-like structure is herein approximately zero. The eigenvalues for the eigenvectors perpendicular to this direction are similar to one another and much smaller than zero for cylindrical vessel-like structures.

Cluster-like or areal structures lead to significantly different eigenvalue combinations, so that on the basis of the eigenvalues, the vesselness measure can be established. For this purpose, different specific calculations are known. The previously cited publication by Y. Sato et al. Proposes, for example, the following vesselness measure λ123:

$${λ}_{123} = \{ \begin{matrix}\vert {λ}_{3} \vert {( \frac{{λ}_{2}}{{λ}_{3}} )}^{{γ}_{23}} {( 1 + \frac{{λ}_{1}}{\vert {λ}_{2} \vert} )}^{{γ}_{12}} , & if {λ}_{3} < {λ}_{2} < {λ}_{1} \leq 0 \\ \vert {λ}_{3} \vert {( \frac{{λ}_{2}}{{λ}_{3}} )}^{{γ}_{23}} {( 1 - α \frac{{λ}_{1}}{\vert {λ}_{2} \vert} )}^{{γ}_{12}} , & if {λ}_{3} < {λ}_{2} < 0 < {λ}_{1} < \frac{\vert {λ}_{2} \vert}{α} \\ 0 , & in other cases\end{matrix}$$

By way of the selection of the variables α, γ12 and γ23, the selectivity of the vesselness measure can be adjusted. A should herein be selected to be between 0 and 1 and γ12 and γ23 should be selected to be greater than or equal to zero. By way of higher values of γ23, false detections of areal structures as linear structures can be suppressed and by way of higher values of α and γ12, the value of the vesselness measure for noise-like structures and/or one-dimensional structures can be reduced. Good results are typically achieved, for example, with α=0.25 and γ12=γ23=1, wherein dependent upon the specific application case, a variation of this value can lead to a further improvement in the recognition of vessel-like structures.

Further possible implementation details for carrying out the calculation described, approaches for approximate calculation of the matrix entries for the reduction of the required computation time and an extension of the described approach for multiscalar identification of vessel-like structures by establishing the vesselness measure for different values of the variance σ of the Gaussian distribution are discussed, for example, in the publication by Y. Sato et al. Cited above and therefore do not need to be repeated.

The obtaining of input image data can comprise the acquisition of the input image data, that is, for example, the performance of a magnetic resonance angiography or another measurement protocol for medical imaging. However, it is also possible that the acquisition of the input image data is provided before the method and is not part of the method. For example, the input image data can be retrieved from a server or read out from an internal memory store of a processing apparatus carrying out the method. The input image data can be, in particular, unmediated scan data and/or three-dimensional image data reconstructed therefrom. It is, however, also possible that the image data is already the result of a pre-processing, that is for example, a prior filtration or scaling.

The filter algorithm can establish the image value of a respective filtered voxel of the filtered image data, dependent, in each case, upon the image values of a plurality of the input image voxels, wherein for establishing the image values of at least two of the filtered voxels, mutually different parameter values are used for the filter parameter or at least one of the filter parameters. Thus, in the method according to an embodiment of the present invention, a locally differing parameterization preferably takes place. Thus, the filtration can be adapted to the locally recognized vessel structure and thus the image quality can be further improved.

The differences in the local parameterization of the filtration can depend exclusively upon the local vesselness measure and/or the vesselness map. In addition or alternatively, however, the directions of recognized vessel-like structures can be taken into account in order to select, for example, the direction in which a particularly strong or a particularly slight filtration takes place by way of an anisotropic filter, as described in more detail below.

A subset of the input image voxels which, according to its vesselness measure is respectively part of a vessel-like structure, can be selected, wherein the parameter value of the filter parameter or at least one of the filter parameters for the respective filtered voxel can be selected dependent upon whether an input image voxel associated with the respective filtered voxel is an element of the subset and/or how far the associated input image voxel is separated from the closest input image voxel which is an element of the subset. The input image voxel associated with the respective filtered voxel can be, in particular, that input image voxel which is situated in the input image data at the same position at which the associated filtered voxel is situated in the filtered image data. Expressed differently, therefore, input image voxels situated at the same position and/or at the same coordinates and filtered voxels are associated with one another.

By way of the selection of the filter parameters selected to be locally different as described above, for example, in regions that are relatively remote from vessel-like structures, a strong filtration can be used, whereby disturbances in the background region are robustly suppressed. In the region of vessel-like structures and/or with increasing closeness thereto, a less strong filtration, for example, by way of adapting the width of a filter kernel or of diffusion constants of a diffusion filter, can be used in order to prevent or minimize a blurring of vessel borders or similar undesirable filtration results.

Preferably, an anisotropic filter algorithm is used, the filter parameter of which is an anisotropy parameter or the filter parameters of which comprise at least one anisotropy parameter, wherein the respective anisotropy parameter controls a degree and/or a direction of the anisotropy of the filter algorithm, so that for at least some of the possible parameter values of the anisotropy parameter, the filter algorithm has a different filter behavior for each of two spatial directions.

In order to achieve a high-quality representation of the vessel-like structure, a strong filtration perpendicularly to the vessel wall and/or to the longitudinal direction, at least for filtered voxels which lie close to the transition region between the background and vessel structures should be avoided. This can be achieved by way of the process described in that the anisotropy of the filter algorithm is specified by way of the choice of suitable parameter values such that a less strong filtration takes place perpendicularly to the longitudinal direction for corresponding filtered voxels. For example, this can be achieved by way of the selection of a narrower filter kernel or by a suitable selection of diffusion constants of a diffusion filter.

As explained above, in order to calculate the respective vesselness measure as an intermediate step, a value matrix associated with the respective input image voxel can be established, wherein the respective vesselness measure is established from eigenvalues of the respective value matrix.

As set out above, the value matrix can be established by way of a convolution of the Hessian matrix of a Gaussian kernel or of the Hessian matrix of another filter kernel with the input image data.

In order to establish the image values of all the filtered voxels of the filtered image data or of a subgroup of the filtered voxels, a parameter value of the anisotropy parameter or of at least one of the anisotropy parameters can be used which is respectively established dependent upon at least one eigenvector of the value matrix that is associated with a respective selected input image voxel. As mentioned above, merely an approximate calculation of the value matrix inputs can also take place.

Since the convolution and derivative operations are interchangeable and the second derivative of the image values of the input image data typically barely deviates from 0 along a vessel-like structure, whereas the second derivative perpendicularly to the longitudinal direction of the vessel-like structure deviates significantly from 0, the established eigenvectors specify the longitudinal direction of the vessel-like structure and/or the directions perpendicularly thereto when the selected input image voxel lies on a vessel-like structure. Thus, for example, a stronger filtration along the vessel-like structure can take place than perpendicularly to its wall, so that despite good noise suppression, the borders of the vessel-like structure remain clearly recognizable.

One of the input image voxels can be fixedly associated with each of the filtered voxels, so that the input image voxel selected for determining the anisotropy parameter of the respective filtered voxel is selected from the or a subset of the input image voxels which is selected on the basis of the vesselness measure, dependent upon the spacing of the respective input image voxel of the subset from the input image voxel associated with the respective filtered voxel.

By way of the procedure described, the longitudinal direction of a vessel-like structure which is mapped by way of the associated input image voxel and thus also the filtered voxel and/or which is located close to the associated input image voxel can be taken into account in order, in particular, as described above, to achieve a stronger filtration in the longitudinal direction of this structure than perpendicularly to the longitudinal direction. By this mechanism and/or means, in particular, a strong blurring and/or softening of the transition between the vessel and the background can also be prevented, even with a relatively strong overall filtration for noise suppression.

The subset of the input image voxels can comprise, in particular, exclusively those input image voxels for which the value of the vesselness measure exceeds a threshold value and/or are classified as mapping a vessel-like structure. From this subset, in particular, the input image voxel, the spacing of which from the input image voxel associated with the respective filtered voxel is a minimum can be selected. If the input image voxel associated with the respective filtered voxel is itself an element of the subset, in particular, this associated input image voxel is selected as the selected input image voxel.

The subgroup of the filtered voxels for which the parameter value of the anisotropy parameter is established dependent upon the respective eigenvector comprises, in an advantageous embodiment, exclusively filtered voxels, the respective associated input image voxel of which is either itself an element of the selected subset of the input image voxels or has a spacing from at least one input image voxel of this subset that is smaller than a specified spacing limit value or corresponds thereto.

For filtered voxels, the associated input image voxels of which are sufficiently far removed from each recognized vessel-like structure, an isotropic filtration can take place and/or the at least one anisotropy parameter can have a fixedly specified value or can vary, for example, dependent upon the spacing from the closest vessel-like structure without taking account of their longitudinal direction. By way of the procedure described, the direction of the extent of the vessel-like structure is taken into account during the filtration, in particular, only if it is also actually relevant, that is for example, if due to a relatively low spacing of the filtered voxel from such a structure, image values from within the structure are and/or would be taken into account during the filtration if the at least one anisotropy parameter were not adapted. This enables, for example, a strong filtration to be carried out in all directions at a sufficient spacing from the vessel-like structure and thus to suppress disturbances in the background of the image significantly better, wherein in the region of and/or close to vessels, the anisotropy of the filtration can be adapted to the course of the vessel.

The three-dimensional medical input image data can be based upon a magnetic resonance angiography without contrast medium administration and/or at a field strength of the main magnet field of less than 1.5 T or less than 1 T. In the case of a magnetic resonance angiography without contrast medium administration and/or at low field strengths, typically, significantly lower signal-to-noise ratios are achieved than would be the case with the use of contrast medium and/or when using stronger fields. Therefore, in these application domains, a high-quality filtration such as can be achieved with the method described, is particularly relevant.

Following the use of the filter algorithm, the image values of the filtered voxels of the filtered image data can be scaled in order to provide scaled image data, wherein a respective scaling factor used for scaling the respective filtered voxel is specified, in each case, dependent upon at least one of the vesselness measures. The scaling can serve, apart from the use of the filtered image data in place of the original input image data, as input data which corresponds to the scaling of the image values of the input image voxels as described above and serve the same purpose, that is, in particular, for background suppression. Features described above for scaling the input image data can be transferred, with the aforementioned advantages, also to the scaling of the filtered image data.

Alternatively, the filter algorithm can be applied to the scaled image data in order to provide filtered image data, wherein the parameter value of at least one filter parameter of the filter algorithm is specified dependent upon at least one of the vesselness measures. Apart from the use of the scaled image data as input data, the filtration of the scaled image data can correspond to the filtration of the input image data as described above. Features described above for filtration of the input image data can be transferred with the advantages mentioned there to the filtration of the scaled image data.

The above-described filtration and scaling, respectively dependent upon the at least one vesselness measure, can thus also be combined and/or carried out in arbitrary sequence one after the other, in order to combine the advantages described in relation to the two approaches and, for example, to achieve a particularly robust background suppression.

As the filter algorithm, an establishing algorithm trained by machine learning can be used, wherein as the input variables of the trained establishing algorithm, both the image values of the input image voxels and also the filter parameters can be used.

In general, a trained function maps cognitive functions which humans associate with other human brains. By way of training based upon training data (machine learning), the trained function is capable of adapting itself to new circumstances and of detecting and extrapolating patterns.

By this mechanism and/or means, corresponding algorithms can be capable, for example, of taking account of image contents during noise suppression if they have been trained with suitable training data. A training can take place, for example in that in the respective training dataset, mappings of the same examination object, that is for example, a phantom or a patient with different main magnet fields are available, so that the algorithm can be trained by way of a per se known supervised learning so as to reduce higher levels of noise in the scan dataset recorded with a lower field such that the result formed is as close as possible to a scan dataset recorded with a higher field. Alternatively or additionally, for example, high-quality scan data can be provided with additional noise in order to obtain a modified scan dataset and the algorithm can be trained such that a deviation of the modified scan data from the high-quality scan data used as reference data is minimized.

Corresponding approaches to training filter algorithms by way of machine learning are per se known. These approaches can be extended in that the algorithm to be trained and therefore also the trained algorithm additionally processes the filter parameters established as described above, which can concern, in particular, the vesselness measures of the input image voxels and/or the value matrices underlying them and/or values established from the variables in question.

An additional information item can be provided to the algorithm, both in the context of the training as well as on its application after the training, specifically the filter parameters, which defines the vessel structure contained in the input image data and/or in the part of the respective training dataset used as input image data, that is in particular, the presence of a vessel-like structure in a particular input image voxel and/or the direction of a vessel structure in the respective voxel. By way of this additional knowledge, a significantly faster and simpler training of the filter algorithm can be achieved and/or the image quality achieved can be further improved.

In general, parameters of a trained function can be adapted by way of training. In particular, supervised learning, semi-supervised learning, unsupervised learning, reinforcement learning and/or active learning can be used. In addition, representation learning (also known as “feature learning”) can be used. The parameters of the trained function can be adapted, in particular, iteratively by way of a plurality of training steps.

A trained function can comprise, for example, a neural network, a support vector machine (SVM), a decision tree and/or a Bayesian network, and/or the trained function can be based upon k-means clustering, Q-learning, genetic algorithms and/or assignment rules. In particular, a neural network can be a deep neural network, a convolutional neural network (CNN) or a deep CNN. Furthermore, the neural network can be an adversarial network, a deep adversarial network and/or a generative adversarial network (GAN).

As described above, the vesselness measures of the input image voxels and/or a three-dimensional vesselness map established therefrom can be used as filter parameters or as part of the filter parameters for the trained establishing algorithm. Thus it can already be communicated, via the filter parameters, to the trained establishing algorithm which structures, specifically the vessel-like structures known on the basis of the vesselness measure, should remain unimpaired in the filtration. The machine learning can thus be used to train the filter algorithm in a targeted manner to disturb vessel-like structures that are defined by the filter parameters as little as possible.

On the basis of the vesselness measures, for example, an unsupervised learning can be implemented additionally or alternatively to a supervised learning. Herein, for example, in the context of the training by way of a downstream algorithm, it can be evaluated to what extent and/or with what contrast level vessel-like structures that are described by the vesselness measure and/or the three-dimensional vesselness map are diverted into the filtered image data and/or to what extent a noise and/or high-frequency signal portions outside the vessel-like structure become suppressed.

In general, an embodiment of the present invention therefore relates to a computer-implemented method for parameterizing an establishing algorithm for providing a filter algorithm by way of machine learning, in particular for use in the computer-implemented method according to an embodiment of the present invention as described above, wherein for three-dimensional medical input image data used in the context of the training, vesselness measures for at least portions of the input image voxels of the input image data and/or a three-dimensional vesselness map associated with the input image data are utilized. The establishment of the vesselness measures and/or of the vesselness map can take place as described above, wherein this establishment can be part of the training process or can be placed upstream thereof.

More specifically, an embodiment of the present invention relates to a computer-implemented method for parameterizing an establishing algorithm for providing a filter algorithm, in particular, for use in the computer-implemented method according to an embodiment of the present invention as described above, by way of machine learning, comprising the steps:
specifying a plurality of training datasets respectively defining three-dimensional medical input image data, vesselness measures for at least parts of the input image voxels of the input image data and/or a three-dimensional vesselness map associated with the input image data, wherein the vesselness measure and/or the vesselness map defines a degree of the similarity of a structure mapped in the respective input image voxel to a mapping of a vessel, and target resultant image data,
minimizing a cost function which depends upon a measure for the respective deviation between the resultant image dataset from the target resultant image data for the plurality of training datasets, by way of variation of a plurality of algorithm parameters of the establishing algorithm for determining an optimum parameter set for the algorithm parameters, wherein the respective resultant image dataset results from the application of the establishing algorithm to respective input data which comprises the three-dimensional medical input image data and, firstly, the vesselness measure and/or, secondly, the vesselness map of the respective training dataset.

The establishing of the vesselness measure and/or of the vesselness map can take place in an upstream step. However, it is also possible that, initially, preliminary training datasets are provided which comprise the input image data and the target resultant image data according to which, in the context of the training process for the respective preliminary training dataset, the vesselness measure and/or the vesselness map are initially established, as already described above, in order to specify the training datasets.

An embodiment of the present invention also relates to a processing apparatus which is configured for carrying out the computer-implemented method, according to an embodiment of the present invention, for processing three-dimensional medical input image data and/or for carrying out the computer-implemented method, according to an embodiment of the present invention, for parameterizing an establishing algorithm for providing a filter algorithm. The processing apparatus can be configured, for example, as suitably programmed data processing facilities or the functionality described can alternatively be implemented as at least partially hard-wired. The processing apparatus can be integrated into a medical imaging device (or facility), in particular into a magnetic resonance tomograph or can be configured separately therefrom. It can be implemented, for example, as a workplace computer, a server or a cloud solution.

An embodiment of the present invention additionally relates to a non-transitory computer-readable storage medium storing a computer program with instructions which are configured, on execution on a data processing device (or facility), to carry out the computer-implemented method, according to an embodiment of the present invention, for processing three-dimensional medical input image data and/or for carrying out the computer-implemented method, according to an embodiment of the present invention, for parameterizing an establishing algorithm for providing a filter algorithm.

In addition, an embodiment of the present invention relates to a data carrier which comprises the computer program, according to an embodiment of the present invention.

### BRIEF DESCRIPTION OF THE DRAWINGS

Further advantages and details of the present invention are disclosed by the following exemplary embodiments and the accompanying drawings. In the drawings, schematically:

FIG. 1 shows an exemplary embodiment of a processing apparatus according to the present invention by way of which an exemplary embodiment of the method according to the present invention for processing three-dimensional medical input image data is carried out,

FIG. 2 shows a flow diagram of the method carried out in FIG. 1,

FIG. 3 shows a flow diagram of an exemplary embodiment of the method according to the present invention for parameterizing an establishing algorithm for providing a filter algorithm, and

FIGS. 4 and 5 show exemplary embodiments of establishing algorithms which can be parameterized by the method shown in FIG. 3 and/or are usable as a filter algorithm in the method of FIG. 2.

### DETAILED DESCRIPTION

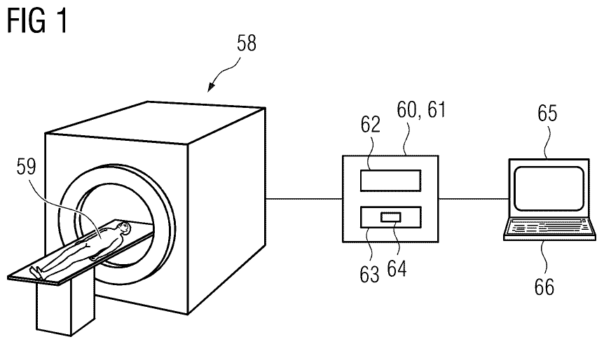

FIG. 1 shows a processing apparatus 61 which serves for processing three-dimensional medical input image data. In the example, the input image data is acquired directly by a medical imaging device (or facility) 58 which, in the example, is a magnetic resonance tomograph and relates to the patient 59. Alternatively, the processed input image data can originate, for example, from a database or from other sources.

The input image data can be, for example, scan data of a magnetic resonance angiography which has been carried out, in particular, at low field strengths of less than 1.5 T and without contrast medium. Therefore, in the example, the input image data is heavily noise-laden and by way of the processing apparatus and/or by way of the method implemented by it for processing the input image data, a filtration of the images is to take place. The filtered image data can then be represented on a display device (or facility) 65 and the representation can be parameterized, for example, via the input device and/or input means 66.

The processing apparatus 61 is implemented in the example by a programmable data processing device (or facility) 60 which comprises a processor 62 and a memory store 63. The memory store 63 stores the computer program 64, the instructions of which carry out the steps of the method described below for processing the input image data.

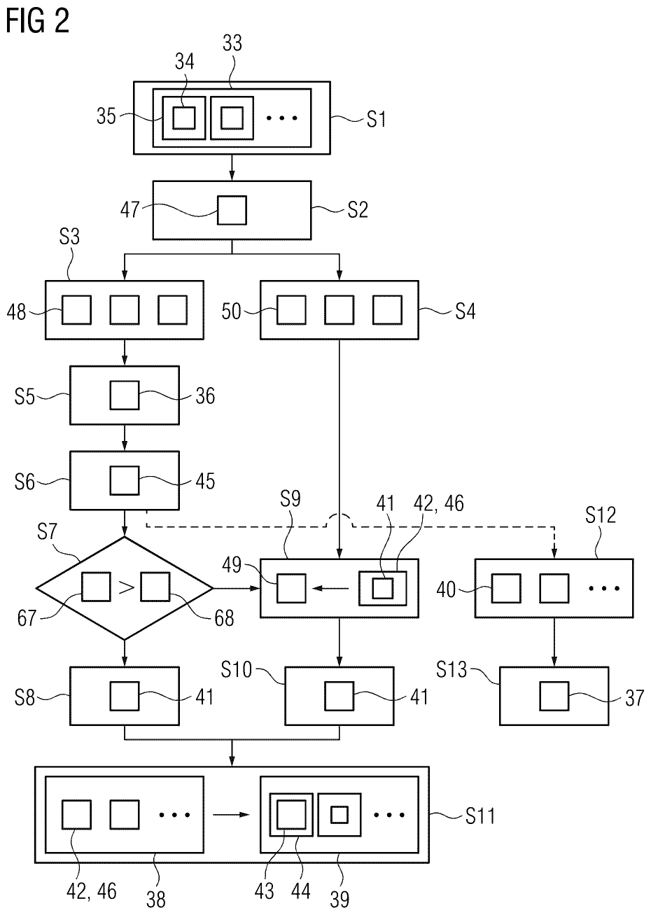

FIG. 2 shows a flow diagram of the method implemented by the processing apparatus 61 for processing the input image data 33. FIG. 2 shows, in detail, an advantageous configuration of the method, so that for better understanding, an overview of the central processing steps will first be set out.

Firstly, in step S1, the input image data 33 is obtained. This defines image values 34 for a large number of input image voxels 35. Step S1 can herein comprise the acquisition of the input image data 33 by the imaging device 58 or merely the receiving of the input image data 33 or the reading out of the input image data 33 from the memory store 63 if it is already stored locally.

By way of the steps S2, S3 and S5, a respective vesselness measure 36 for each of the input image voxels 35 is determined which defines an established degree of similarity of a structure mapped in the respective input image voxel 35 to a mapping of a vessel. An example for this establishing will be set out in greater detail further below.

Subsequently, in steps S8 to S10, parameter values 41 of filter parameters 42 of a filter algorithm 38 are specified locally for the respective voxel, dependent upon the vesselness measures 36, which will also be described in greater detail further below.

In step S11, a filter algorithm 38 is then applied to the input image data 33 in order to provide filtered data 39, wherein the filter parameters 42 are specified by the previously established parameter values 41.

The filter algorithm 38 therein establishes the image value 43 of a respective filtered voxel 44 of the filtered image data 39 dependent upon the image values 34 of a plurality of the input image voxels 35, for example, by using a filter kernel or by way of a diffusion filter. For the establishing of the image values 43 of at least two of the filtered voxels 44, herein mutually different parameter values 41 are used for at least one of the filter parameters 42, so that the locally different parameterizing as described above results. This can be implemented, for example, in that locally different filter kernels and/or a position-dependent convolution kernel are used or in that, for example, a diffusion filter with locally differing parameterizing is utilized.

Alternatively or in addition to the filtration described, in the steps S12 and S13, a scaling of the image values 34 of the input image voxels 35 can take place to provide scaled image data 37. For this purpose, in step S12, for each of the input image voxels 35, an associated scaling factor 40 can firstly be established. The scaling factor 40 can be selected such that for input image voxels the vesselness measure 36 of which indicates their belonging to the vessel system, it is significantly greater than for input image voxels 35 for which this is not the case. For example, the scaling factors can be proportional to the vesselness measure 36 and/or can be selected to be very small, if the vesselness measure exceeds a limit value.

In step S13, the image value 34 of each input image voxel 35 can be multiplied by the scaling factor 40 established for this input image voxel 35 in order to generate scaled image data 37. By this mechanism and/or means, the vessel system mapped in the input image data is emphasized and a background is suppressed. The scaled image data 37 thereby established can also be used in step S11 in place of the input image data 33 as input data of the filter algorithm 38. A corresponding scaling would additionally be possible in an alternative embodiment or alternatively also for the already filtered voxels 44.

In the following, the specific implementation of the steps discussed only in outline above are described in greater detail for the method configuration shown by way of example in FIG. 2.

Herein, after obtaining the input image data 33 in step S1, in step S2, for each of the input image voxels 35 of the input image data 33, firstly, an associated value matrix 47 is established in that a convolution of the Hessian matrix of a Gaussian kernel is calculated with the input image data 33. A specific calculation of the matrix inputs Iab has already been set out in the general part of the description.

Subsequently, in step S3, the eigenvalues 48 of the value matrix 47 which are identified in the formulae set out in the general part as λ1, λ2 and λ3, and in step S4, the eigenvectors 50 associated with the eigenvalues 48 are established. Algorithms for establishing eigenvalues and eigenvectors of matrices belong to the standard mathematical repertoire and therefore will not be described here.

From the eigenvalues 48, in step S5, the vesselness measures 36 which are designated λ123 above are calculated as described above.

In step S6, in the example, at least one subset 45 of the input image voxels 35 is selected which, according to their vesselness measure 36, are each part of a vessel-like structure. For example, all the input image voxels 35, the vesselness measure 36 of which exceeds a specified limit value, can be accepted into this subset 45.

In step S7, for each filtered voxel 44 for which in step S11 an image value 43 is to be established, it is then tested whether its spacing and/or the spacing 67 of the input image voxel 35 associated with it, which is situated at the same position as the associated filtered voxel 44, from the nearest input image voxel that is part of the subset 45, exceeds a specified spacing limit value 68.

If this is the case, it can be assumed therefrom that a relatively strong filtration in the region of this voxel does not impair the representation of imaged vessel structures, since the voxel is sufficiently far removed from voxels of the subset 45 and thus from recognized vessel-like structures.

Therefore, in step S8, for example, for such voxels, fixedly specified parameter values 41 for the filter parameters 42 can be used which can lead, for example, to a relatively strong, at least substantially isotropic, filtration.

For the subgroup 49 of the filtered voxels the spacing 67 of which is smaller than or equal to the spacing limit value 68, such a strong filtration could however potentially lead to a blurring of vessel boundaries and/or to some other impairment of the vessel representation, which should be prevented. Therefore, another parameterizing of the filtration should take place.

In a simplified variant of the method described, an isotropic filtration could also take place for the filtered voxels of the subgroup, wherein by way of another selection of the parameter values 41 dependent, in particular, upon the spacing 67, close to the vessel structure, a weaker filtration, for example, by way of lower diffusion constants and/or a narrower filter kernel, could be achieved.

In the example shown, however, an anisotropic filter algorithm 38 is used, the filter parameters 42 of which comprise anisotropy parameters 46, and which controls a degree and a direction of the anisotropy 42 of the filter algorithm 38. As discussed above in the general part, it can be achieved, by way of suitable selection of the parameter values 41 of the anisotropy parameters 46 in the step S9, that for a direction perpendicular to the longitudinal direction of a vessel-like structure, a weaker filtration takes place than in the longitudinal direction.

For the selection of a suitable parameterizing, in the example, firstly, for the respective filtered voxel 44 of the subgroup 49, the closest input image voxel 35 which belongs to the subset 45 and thus apparently maps a vessel-like structure is selected. If the input image voxel 35 associated with the filtered voxel 44 is itself a member of the subset, then this is selected.

The eigenvectors 50 established in step S4 of the value matrix of the respectively selected input image voxel 35 define a local longitudinal direction in which the vessel-like structure apparently extends. Herein, the eigenvector 50 which is associated with the quantitatively smallest eigenvalue corresponds to the longitudinal direction of the closest portion in the vessel structure so that a relatively strong filtration in the direction specified by this eigenvector is possible, for example, by the use of a relatively broad filter kernel.

In the simplest case, by way of a suitable selection of an anisotropy parameter, the filter strengths in both directions perpendicular to the longitudinal direction of the closest vessel-like structure can be reduced in relation thereto. The degree of the reduction can herein be dependent upon the spacing 67.

The result of the filtration can potentially, however, be further improved if a strong filtration also takes place perpendicularly to the plane, which is formed by the longitudinal direction of the vessel-like structure and the connecting line between the respective filtered voxel and/or the input image voxel associated with it and the input image voxel selected for this filtered voxel, since for this direction, for example, no overlap of a filter kernel with the closest vessel-like structure is to be expected.

Following the described selection of the parameter values 41 of the anisotropy parameters 46 in step S9, in step S10, further parameter values 41 for the remaining filter parameters 42 which do not influence the anisotropy can be selected. This selection can take place, for example, dependent upon the spacing 67 in order to use an overall weaker filtration independently of the described anisotropy close to or on vessel-like structures, than would be achieved by way of the parameter values 41 specified in step S8 for voxels remote from the vessel-like structure.

Then, in step S11, as described above, the filter algorithm 38 parameterized by way of the parameter values 41 can be applied to the input image data 33 or the scaled image data 37 in order to create the filtered image data 39.

By way of the procedure described, the local parameterizing of the filtration is adapted in order to achieve weaker filtration, at least in the direction perpendicular to the longitudinal direction of the closest vessel-like structure, close to and/or on vessel-like structures that are recognized on the basis of the vesselness measure 48, than is used in regions that are spaced further from the vessel-like structure. Thus, in pure background regions that are remote from vessel-like structures, strong filtrations can be used that, without this weakening of the filtration dependent upon the vesselness measure and/or taking account of the longitudinal direction of the closest vessel-like structure, would severely blur the borders of the vessel-like structure and would thus be unsuitable for a high-quality representation of vessel-like structures. Thus at least substantially without impairing the sharpness of the representation of the vessel-like structure, a significantly improved noise suppression, in particular in the background region, can be achieved.

It is per se known that, for example, for filters based upon a filter kernel and for diffusion filters, the filter behavior can be adapted by way of the specification of parameter values 41 for filter parameters 42, specifically for anisotropy parameters 46. For example, an extent of a filter kernel can be scaled and/or diffusion constants can be adapted. These filter algorithms can therefore be used without difficulty in the method according to one or more embodiments of the present invention.

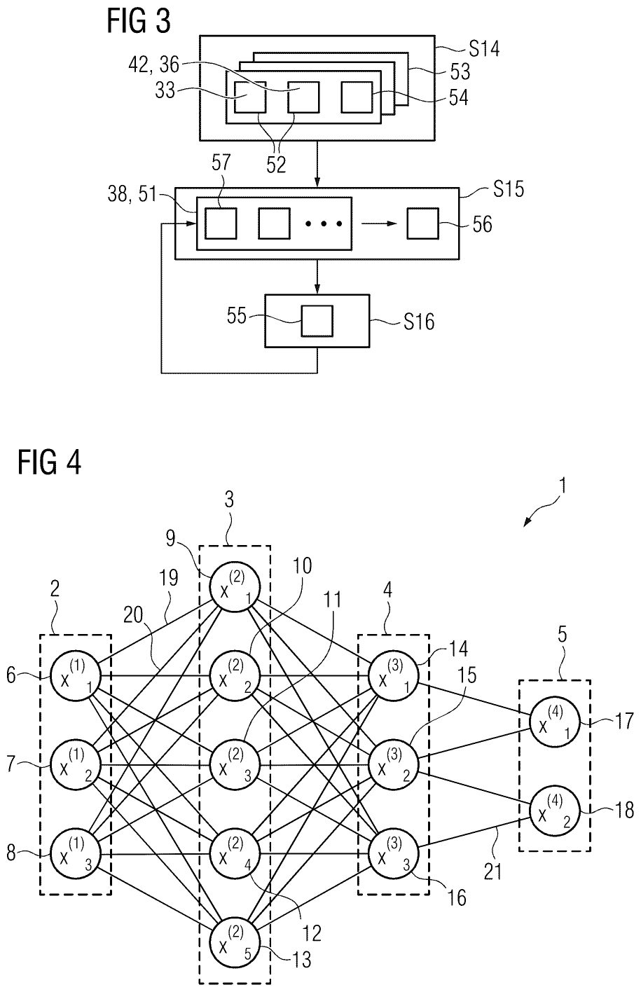

It is however also possible that an establishing algorithm 51 trained by way of machine learning is used as the filter algorithm 38, for which purpose an exemplary training is shown schematically in FIG. 3. In the example, it is herein assumed that as input variables 52 of the trained establishing algorithm 51, both the image values 34 of the input image voxel 35 and also the filter parameters 42 are used. In the example according to FIG. 2, herein, the vesselness measures 48 of the input image voxel 35 are used directly as filter parameters 42 which can be established on the basis of the image values 34, for example, according to the steps S1 to S3 described above.

In step S14, initially a plurality of training datasets 53 are specified, on the basis of which a training is to be performed. The training datasets 53 each comprise three-dimensional medical input image data 33 and target resultant image data 54. The target resultant data items 54 each specify a desired result to which a resultant image dataset 56, which results on application of the trained establishing algorithm 51 and thus of the filter algorithm 38 to the input data 52 of the respective training dataset, is to correspond as exactly as possible.

As described above in the general part, pairs of input image data 33 and target resultant image data 54 can be established, for example, in that for acquisition of the input image data 33 and the target resultant image data 54, mutually different field strengths of the main field of a magnetic resonance tomograph are used and/or in that the image quality in the input image data 33 is artificially worsened as compared with the target resultant image data 54, for example, by way of an additive noise. As described, the filter parameters 42 can be established on the basis of the respective input image data 33 and, in the example, correspond to the vesselness measures 36.

In step S15, the establishing algorithm 51 which is initially provisionally parameterized by way of the algorithm parameters 57 is applied to the input data 52, that is, in the example, to the input image data 33 and their vesselness measures 36, in order to establish a resultant image dataset 56.

Then, in step S16, a cost function 55 is applied which depends upon a measure for the respective deviation of the resultant image dataset 56 from the target resultant image data 54 for a plurality of training datasets 53. For example, the individual deviation measures can be summed.

By way of variation of the algorithm parameters 57, the cost function 55 is then minimized iteratively in order to determine an optimum parameter set for the algorithm parameters 57. Herein, in particular, known approaches to error backpropagation can be used. Overall, in the example, a supervised learning on the basis of the training datasets 53 is therefore implemented.

The establishing algorithm 51 parameterizing according to the optimum parameter set can then be used as a filter algorithm 38.

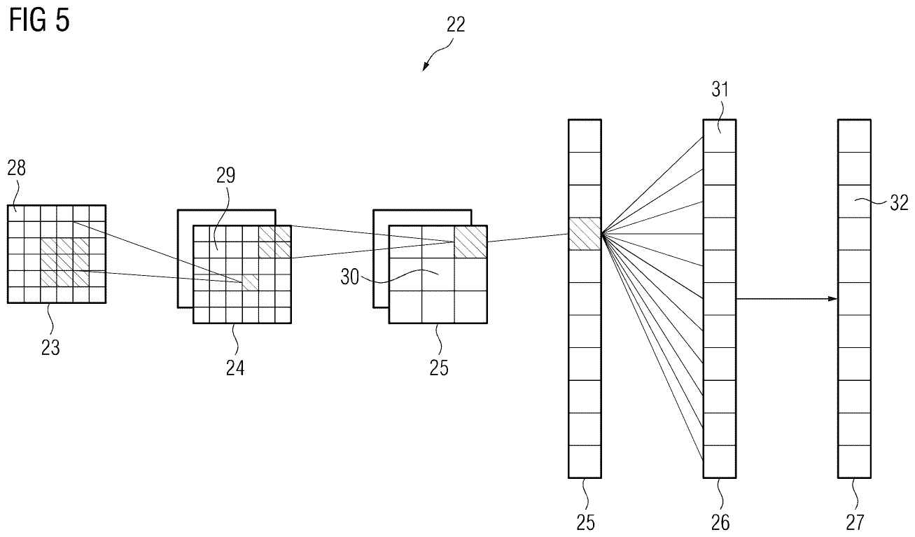

Possible structures of establishing algorithms which can be used herein are described below with additional reference to FIGS. 4 and 5. In the interests of a readily comprehensible representation, herein, by way of example, neural networks with a relatively small number of nodes are discussed. In real applications, typically higher node, and therefore parameter, counts are used. Other structures of the neural nets and/or fundamentally different algorithms of machine learning, examples of which were given in the general part, are also usable.

FIG. 4 shows an exemplary embodiment of an artificial neural network 1. Other expressions for the artificial neural network 1 are “neural network”, “artificial neural net” or “neural net”.

The artificial neural network 1 comprises nodes 6 to 18 and edges 19 to 21, wherein each edge 19 to 21 is a directed connection from a first node 6 to 18 to a second node 6 to 18. In general, the first node 6 to 18 and the second node 6 to 18 are different nodes 6 to 18, but it is also conceivable that the first node 6 to 18 and the second node 6 to 18 are identical. For example, in FIG. 4, the edge 19 is a directed connection from the node 6 to the node 9 and the edge 21 is a directed connection from the node 16 to the node 18. An edge 19 to 21 from a first node 6 to 18 to a second node 6 to 18 is designated an “ingoing edge” for the second node 6 to 18 and as an “outgoing edge” for the first node 6 to 18.

In this exemplary embodiment, the nodes 6 to 18 of the artificial neural network 1 can be arranged in layers 2 to 5, wherein the layers can have an intrinsic order which is introduced by the edges 19 to 21 between the nodes 6 to 18. In particular, edges 19 to 21 can only be provided between adjacent layers of nodes 6 to 18. In the exemplary embodiment shown, there exists an input layer 2 which has only the nodes 6, 7, 8, in each case without an ingoing edge. The output layer 5 comprises only the nodes 17, 18 each without outgoing edges, wherein furthermore, hidden layers 3 and 4 lie between the input layer 2 and the output layer 5. In the general case, the number of hidden layers 3, 4 can be selected arbitrarily. The number of nodes 6, 7, 8 of the input layer 2 typically corresponds to the number of input values into the neural network 1 and the number of the nodes 17, 18 in the output layer 5 typically corresponds to the number of the output values of the neural network 1.

In particular, a (real) number can be assigned to the nodes 6 to 18 of the neural network 1. Therein, x(n)i denotes the value of the i-th node 6 to 18 of the n-th layer 2 to 5. The values of the nodes 6, 7, 8 of the input layer 2 are equivalent to the input values of the neural network 1, while the values of the nodes 17, 18 of the output layer 5 are equivalent to the output values of the neural network 1. Furthermore, each edge 19, 20, 21 can be allocated a weight in the form of a real number. In particular, the weight is a real number in the interval [−1, 1] or in the interval [0, 1]. Therein, w (m, n) i, j denotes the weight of the edge between the i-th nodes 6 to 18 of the m-th layer 2 to 5 and the j-th nodes 6 to 18 of the n-th layer 2 to 5. Furthermore, the abbreviation W_{i,j}⁽ⁿ⁾ is defined for the weight W_{i,j}^{(n,n+1)}.

In order to calculate output values of the neural network 1, the input values are propagated by the neural network 1. In particular, the values of the nodes 6 to 18 of the (n+1)-th layer 2 to 5 can be calculated on the basis of the values of the nodes 6 to 18 of the n-th layer 2 to 5 with

$${x}_{j}^{( n + 1 )} = f ( {\sum }_{i} {x}_{j}^{( n )} \cdot {w}_{i , j}^{( n )} )$$

Therein, f is a transfer function which can be designated the activation function. Known transfer functions are step functions, sigmoid functions (for example, the logistic function, the generalized logistic function, the hyperbolic tangent, the arctangent, the error function, the smoothstep function) or rectifiers. The transfer function is substantially used for normalizing purposes.

In particular, the values are propagated layerwise by way of the neural network 1, wherein values of the input layer 2 are given by way of the input data of the neural network 1. Values of the first hidden layer 3 can be calculated on the basis of the values of the input layer 2 of the neural network 1, and values of the second hidden layer 4 can be calculated on the basis of the values in the first hidden layer 3, etc.

In order to be able to specify the values W_{i,j}⁽ⁿ⁾ for the edges 19 to 21, the neural network 1 must be trained using training data. In particular, training data comprises training input data and training output data which are denoted below as ti. For a training step, the neural network 1 is applied to the training input data in order to establish calculated output data. In particular, the training output data and the calculated output data comprise a number of values, wherein the number is determined as the number of the nodes 17, 18 of the output layer 5.

In particular, a comparison between the calculated output data and the training output data is used to adapt recursively the weights within the neural network 1 (“back-propagation algorithm”). In particular, the weights can be amended according to

$${w}_{i , j}^{′ ( n )} = {w}_{i , j}^{( n )} - γ \cdot {δ}_{j}^{( n )} \cdot {x}_{i}^{( n )}$$

wherein γ is a learning rate and the numbers δⱼ⁽ⁿ⁾ can be calculated recursively according to

$${δ}_{j}^{( n )} = ( {\sum }_{k} {δ}_{j}^{( n + 1 )} \cdot {w}_{i , j}^{( n + 1 )} ) \cdot {f}^{′} ( {\sum }_{i} {x}_{i}^{( n )} \cdot {w}_{i , j}^{( n )} )$$

on the basis of δⱼ⁽ⁿ⁺¹⁾ when the (n+1)-th layer is not the output layer 5, and

$${δ}_{j}^{( n )} = ( {x}_{k}^{( n + 1 )} - {t}_{j}^{( n + 1 )} ) \cdot {f}^{′} ( {\sum }_{i} {x}_{i}^{( n )} \cdot {w}_{i , j}^{( n )} )$$

if the (n+1)-th layer is the output layer 5, wherein “f” is the first derivative of the activation function and yⱼ⁽ⁿ⁺¹⁾ is the comparative training value for the j-th nodes 17, 18 of the output layer 5.

An example will now also be given for a convolutional neural network (CNN), making reference to FIG. 5. It should be noted here that the expression “layer” is used therein in a slightly different way than for classical neural networks. For a classical neural network, the expression “layer” merely indicates the set of nodes which form a layer and therefore a particular generation of nodes. For a convolutional neural network, the expression “layer” is often used as an object which actively changes data, in other words as a set of nodes of the same generation and the set of either ingoing or outgoing edges.

FIG. 5 shows an exemplary embodiment of a convolutional neural network 22. In the exemplary embodiment shown, the convolutional neural network 22 comprises an input layer 23, a convolutional layer 24, a pooling layer 25, a completely connected layer 26 and an output layer 27. In alternative embodiments, the convolutional neural network 22 can contain a plurality of convolutional layers 24, a plurality of pooling layers 25 and a plurality of completely connected layers 26, exactly like other types of layers. The sequence of the layers can be selected as desired, wherein typically, completely connected layers 26 form the last layers before the output layer 27.

In particular, within a convolutional neural network 22, the nodes 28 to 32 of one of the layers 23 to 27 can be understood as being arranged in a d-dimensional matrix or as a d-dimensional image. In particular, in the two-dimensional case, the value of a node 28 to 32 can be denoted with the indices i, j in the n-th layer 23 to 27 as x(n)[i, j]. It should be noted therein that the arrangement of the nodes 28 to 31 of a layer 23 to 27 has no effect on the calculations within the convolutional neural network 22 as such, since these effects are produced entirely by the structure and the weights of the edges.

A convolutional layer 24 is distinguished, in particular, in that the structure and the weights of the ingoing edges form a convolution operation on the basis of a particular number of kernels. In particular, the structure and the weights of the ingoing edges can be selected such that the values Xₖ⁽ⁿ⁾ of the nodes 29 of the convolutional layer 24 are established as a convolution Xₖ⁽ⁿ⁾=Kₖ*X⁽ⁿ⁻¹⁾ on the basis of the values X⁽ⁿ⁻¹⁾ of the nodes 28 of the preceding layer 23, wherein the convolution*in the two-dimensional case can be defined as

$${x}_{k}^{( n )} [ i , j ] = ( {K}_{k} * {x}^{( n - 1 )} ) [ i , j ] = {\sum }_{{i}^{′}} {\sum }_{{j}^{′}} {K}_{k} [ {i}^{′} , {j}^{′} ] \cdot {x}^{( n - 1 )} [ i - {i}^{′} , j - {j}^{′} ]$$

Therein, the k-th kernel Kk is a d-dimensional matrix, in this exemplary embodiment a two-dimensional matrix, which is typically small in comparison with the number of the nodes 28 to 32, for example, a 3×3 matrix or a 5×5 matrix. In particular, this implies that the weights of the ingoing edges are not independent, but rather are selected such that they generate the above convolution equation. In the example for a kernel which forms a 3×3 matrix, there exist only nine independent weights (wherein each entry of the kernel matrix represents an independent weight), regardless of the number of nodes 28 to 32 in the corresponding layer 23 to 27. In particular, for a convolutional layer 24, the number of nodes 29 in the convolutional layer 24 is equivalent to the number of nodes 28 in the preceding layer 23 multiplied by the number of convolution kernels.

If the nodes 28 of the preceding layer 23 are arranged as a d-dimensional matrix, the use of the plurality of kernels can be understood as adding a further dimension which is also designated the depth dimension, so that the nodes 29 of the convolutional layer 24 are arranged as a (d+1)-dimensional matrix. If the nodes 28 of the preceding layer 23 are already arranged as a (d+1)-dimensional matrix with a depth dimension, the use of a plurality of convolution kernels can be understood as an expansion along the depth dimension, so that the nodes 29 of the convolutional layer 24 are likewise arranged as a (d+1)-dimensional matrix, wherein the size of the (d+1)-dimensional matrix in the depth dimension is larger by the factor formed by the number of the kernels than in the preceding layer 23.

The advantage of the use of convolutional layers 24 is that the spatially local correlation of the input data can be utilized in that a local connecting pattern is created between nodes of adjacent layers, in particular in that each node has connections only to a small region of the nodes of the preceding layer.

In the exemplary embodiment shown, the input layer 23 comprises thirty-six nodes 28 which are arranged as a two-dimensional 6×6 matrix. The convolutional layer 24 comprises seventy-two nodes 29 which are arranged as two two-dimensional 6×6 matrices, wherein each of the two matrices is the result of a convolution of the values of the input layer 23 with a convolution kernel. In the same way, the nodes 29 of the convolutional layer 24 can be understood as being arranged in a three-dimensional 6×6×2 matrix, wherein the last-mentioned dimension is the depth dimension.

A pooling layer 25 is distinguished in that the structure and the weights of the ingoing edges and the activation function of its nodes 30 define a pooling operation on the basis of a non-linear pooling function f. For example, in the two-dimensional case, the values x(n) of the nodes 30 of the pooling layer 25 can be calculated on the basis of the values x(n+1) of the nodes 29 of the preceding layer 24 as

$${x}^{( n )} [ i , j ] = f ( {x}^{( n - 1 )} [ {id}_{1} , {jd}_{2} ] , … , {x}^{( n - 1 )} [ {id}_{1} + {d}_{1} - 1 , {jd}_{2} + {d}_{2} - 1 ] )$$

In other words, by way of the use of a pooling layer 25, the number of nodes 29, 30 can be reduced in that a number d1×d2 of adjacent nodes 29 in the preceding layer 24 are replaced by a single node 30 which is calculated as a function of the values of said number of adjacent nodes 29. In particular, the pooling function f can be a maximum function, an averaging or the L2 norm. In particular, for a pooling layer 25, the weights of the ingoing edges can be specified and not modified by training.

The advantage of the use of a pooling layer 25 is that the number of nodes 29, 30 and the number of parameters is reduced. This leads to a reduction of the required calculation quantity within the convolutional neural network 22 and thus to control of the overfitting.

In the exemplary embodiment shown, the pooling layer 25 is a max-pooling layer in which four adjacent nodes are replaced with just one single node, the value of which is formed by the maximum of the values of the four adjacent nodes. The max-pooling is applied to each d-dimensional matrix of the preceding layer; in this exemplary embodiment, the max-pooling is applied to each of the two two-dimensional matrices so that the number of the nodes is reduced from seventy-two to eighteen.

A completely connected layer 26 is distinguished in that a plurality, in particular all, of the edges between the nodes 30 of the preceding layer 25 and the nodes 31 of the completely connected layer 26 are present, wherein the weight of each of the edges can be individually adapted. In this exemplary embodiment, the nodes 30 of the preceding layer 25 and of the completely connected layer 26 are shown both as two-dimensional matrices and also as non-coherent nodes (shown as one row of nodes, wherein the number of the nodes has been reduced for better clarity). In this exemplary embodiment, the number of the nodes 31 in the completely connected layer 26 is equal to the number of the nodes 30 in the preceding layer 25. In alternative embodiments, the number of the nodes 30, 31 can be different.

Furthermore, in this exemplary embodiment, the values of the nodes 32 of the output layer 27 are determined in that the Softmax function is applied to the values of the nodes 31 of the preceding layer 26. By applying the Softmax function, the sum of the values of all the nodes 32 of the output layer 27 is one and all the values of all the nodes 32 of the output layer are real numbers between 0 and 1. If the convolutional neural network 22 is used for classifying input data, in particular, the values of the output layer 27 can be interpreted as a probability that the input data falls into one of the different classes.

A convolutional neural network 22 can likewise have a ReLU layer, wherein ReLU is an acronym for “rectified linear units”. In particular, the number of the nodes and the structure of the nodes within a ReLU layer is equivalent to the number of the nodes and the structures of the nodes of the preceding layer. The value of each node in the ReLU layer can be calculated, in particular, by applying a rectifier function to the value of the corresponding node of the preceding layer. Examples of rectifier functions are f(x)=max (0, x), the hyperbolic tangent or the sigmoid function.

Convolutional neural networks 22 can be trained, in particular, on the basis of the back-propagation algorithm. In order to prevent an overfitting, regularization methods can be used, for example, dropout of individual nodes 28 to 32, stochastic pooling, the use of synthetic data, weight decay on the basis of the L1 or L2 norm or maximal norm restrictions.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing device or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been illustrated and described in detail by way of the example embodiments, the present invention is not restricted by the examples disclosed and other variations can be derived therefrom by a person skilled in the art without departing from the protective scope of the present invention.

What is claimed is:

1. A computer-implemented method for processing three-dimensional medical input image data, the computer-implemented method comprising:
obtaining input image data that defines image values for a first number of input image voxels;
establishing a respective vesselness measure for at least parts of the input image voxels, wherein the respective vesselness measure defines an established degree of similarity of a structure mapped in a respective input image voxel to a mapping of a vessel; and
applying a filter algorithm to the input image data for providing filtered image data, or
scaling the image values of the input image voxels for providing scaled image data, wherein
a parameter value of at least one filter parameter of the filter algorithm is dependent upon at least one vesselness measure, or
a respective scaling factor used for scaling a respective image value is dependent upon at least one vesselness measure.

2. The computer-implemented method as claimed in claim 1, wherein
the filter algorithm establishes an image value of a respective filtered voxel of the filtered image data dependent upon the image values of a plurality of the input image voxels, and
for establishing the image values of at least two filtered voxels, mutually different parameter values are used for the filter parameter or at least one of the filter parameters.

3. The computer-implemented method as claimed in claim 2, further comprising:
selecting a subset of the input image voxels that are, according to respective vesselness measures, part of a vessel-like structure, wherein
the parameter value of the filter parameter or at least one of the filter parameters for the respective filtered voxel is selected dependent upon at least one of whether an input image voxel associated with the respective filtered voxel is an element of the subset or how far the associated input image voxel is separated from a closest input image voxel that is an element of the subset.

4. The computer-implemented method as claimed claim 1, wherein
the filter algorithm is an anisotropic filter algorithm,
the at least one filter parameter of the anisotropic filter algorithm includes at least one anisotropy parameter,
a respective anisotropy parameter, among the at least one anisotropic parameter, controls at least one of a degree or a direction of an anisotropy of the anisotropic filter algorithm, such that for at least some possible parameter values of the at least one anisotropy parameter, the anisotropic filter algorithm has a different filter behavior for each of two spatial directions.

5. The computer-implemented method as claimed in claim 1, wherein
establishing a value matrix to calculate the respective vesselness measure as an intermediate step, the value matrix being associated with the respective input image voxel, wherein
the respective vesselness measure is established from eigenvalues of the value matrix.

6. The computer-implemented method as claimed in claim 5, wherein the value matrix is established via a convolution of a Hessian matrix of a Gaussian kernel or of a Hessian matrix of another filter kernel with the input image data.

7. The computer-implemented method as claimed in claim 6, wherein
the at least one filter parameter includes at least one anisotropic filter parameter, and
for establishing the image values of at least a subgroup of filtered voxels of the filtered image data, the parameter value of the at least one anisotropic filter parameter is used, the parameter value being dependent upon at least one eigenvector of the value matrix that is associated with a respective selected input image voxel.

8. The computer-implemented method as claimed in claim 7, wherein
an input image voxel, of the input image voxels, is associated with each of the filtered voxels,
the input image voxel selected for determining the anisotropic filter parameter of a respective filtered voxel is selected from the input image voxels or a subset of the input image voxels which is selected based on the vesselness measure, dependent upon a spacing of the respective input image voxel of the subset from the input image voxel associated with the respective filtered voxel.

9. The computer-implemented method as claimed in claim 8, wherein
the subgroup of filtered voxels includes exclusively filtered voxels, a respective associated input image voxel of which is either an element of the subset of the input image voxels or has a spacing from at least one input image voxel of the subset that is smaller than or corresponds to a threshold spacing limit value.

10. The computer-implemented method as claimed in claim 1, wherein the input image data is based upon a magnetic resonance angiography at least one of without contrast medium usage or at a field strength of a main magnet field of less than 1.5 T.

11. The computer-implemented method as claimed in claim 1, wherein
the image values of filtered voxels of the filtered image data are scaled to provide the scaled image data, and wherein a respective scaling factor used for scaling a respective filtered voxel is, in each case, dependent upon at least one vesselness measure, or
a filter algorithm is applied to the scaled image data to provide filtered image data, wherein a parameter value of at least one filter parameter of the filter algorithm applied to the scaled image data is dependent upon at least one vesselness measure.

12. The computer-implemented method as claimed in claim 1, wherein
an establishing algorithm trained by machine learning is used as the filter algorithm, and
both the image values of the input image voxels and the filter parameter are used as input variables of the establishing algorithm.

13. The computer-implemented method as claimed in claim 12, wherein at least one vesselness measure of the input image voxels or a three-dimensional vesselness map are used as at least one filter parameter or as part of the at least one filter parameter for the establishing algorithm, and wherein the three-dimensional vesselness map is based on vesselness measures of the input image voxels.

14. A computer-implemented method for parameterizing an establishing algorithm for providing a filter algorithm, by way of machine learning, the computer-implemented method comprising:
specifying a plurality of training datasets defining three-dimensional medical input image data,
at least one vesselness measure for at least parts of input image voxels of the three-dimensional medical input image data or a three-dimensional vesselness map associated with the three-dimensional medical input image data, wherein the at least one vesselness measure or the three-dimensional vesselness map defines a degree of similarity of a structure mapped in a respective input image voxel to a mapping of a vessel, and
target resultant image data,
minimizing a cost function that depends upon a measure for a respective deviation between a resultant image dataset from the target resultant image data for the plurality of training datasets, by varying a plurality of algorithm parameters of the establishing algorithm to determine an optimum parameter set for the plurality of algorithm parameters, wherein
the resultant image dataset results from application of the establishing algorithm to respective input data that includes the three-dimensional medical input image data and at least one of the at least one vesselness measure or the three-dimensional vesselness map.

15. A processing apparatus configured to carry out the computer-implemented method as claimed in claim 1.

16. A non-transitory computer-readable medium storing computer-executable instructions that, when executed on a data processing device, cause the data processing device to perform the computer-implemented method as claimed in claim 1.

17. The computer-implemented method as claimed claim 3, wherein
the filter algorithm is an anisotropic filter algorithm,
the at least one filter parameter of the anisotropic filter algorithm includes at least one anisotropic parameter,
a respective anisotropic parameter, among the at least one anisotropic parameter, controls at least one of a degree or a direction of an anisotropy of the anisotropic filter algorithm, such that for at least some possible parameter values of the at least one anisotropy parameter, the anisotropic filter algorithm has a different filter behavior for each of two spatial directions.

18. The computer-implemented method as claimed in claim 17, wherein
establishing a value matrix to calculate the respective vesselness measure as an intermediate step, the value matrix being associated with the respective input image voxel, wherein
the respective vesselness measure is established from eigenvalues of the value matrix.

19. The computer-implemented method as claimed in claim 3, wherein
establishing a value matrix to calculate the respective vesselness measure as an intermediate step, the value matrix being associated with the respective input image voxel, wherein
the respective vesselness measure is established from eigenvalues of the value matrix.

20. The computer-implemented method as claimed in claim 3, wherein
the image values of filtered voxels of the filtered image data are scaled to provide the scaled image data, and wherein a respective scaling factor used for scaling a respective filtered voxel is, in each case, dependent upon at least one vesselness measure, or
a filter algorithm is applied to the scaled image data to provide filtered image data, wherein a parameter value of at least one filter parameter of the filter algorithm applied to the scaled image data is dependent upon at least one vesselness measure.
